(19) United States
(12) Patent Application Publication
(10) Pub. No.: US 2008/0221582 A1
(43) Pub. Date: Sep. 11, 2008

(54) PULMONARY STENT REMOVAL DEVICE

(75) Inventors: Son Gia, San Jose, CA (US); Ajit Nair, Milpitas, CA (US); Andrew Huffmaster, Newark, CA (US); Jeffrey Lee, San Ramon, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: PULMONx, Palo Alto, CA (US)

(21) Appl. No.: 12/041,827

(22) Filed: Mar. 4, 2008

Related U.S. Application Data
(60) Provisional application No. 60/893,051, filed on Mar. 5, 2007.

(51) Int. Cl. A61B 17/58

(52) U.S. Cl. 606/99; 606/108

(57) ABSTRACT

A removal tool for an implanted device, including pulmonary stents, occlusive devices, valved devices, and flow-restrictive devices, is provided. The removal tool includes an elongate tube having a central passage, a slideable inner member within the passage, and a coupling member disposed on the distal end of the inner member. The coupling member of the removal tool includes a distal tip configured to pierce a membrane of the implanted device. The coupling member also includes a coil or a hook configured to engage a support element of the implanted device. A method of removing implanted devices is also provided. A removal tool is placed adjacent to the device, the distal end of the tool is moved to pierce its membrane, a portion of the tool engages the support member, and the distal end of the tool is retracted along with the implanted device.

### CROSS-REFERENCES TO RELATED APPLICATIONS

This application claims the benefit of provisional application Ser. No. 60/893,051 (Attorney Docket No. 017534-004300US), filed Mar. 5, 2007, entitled “Pulmonary Stent Removal Device,” the full disclosure of which is incorporated herein by reference.

### BACKGROUND OF THE INVENTION

1. Field of Invention

The present invention relates to medical devices, methods, systems and kits. More particularly, the present invention relates to devices and methods for the removal of implanted devices that have been positioned within a body lumen, for example, a bronchial passage. Such devices include pulmonary devices which may be occlusive, valved devices, or flow-restrictive.

Chronic obstructive pulmonary disease is a significant medical problem affecting 16 million people or about 6% of the U.S. population. Specific diseases in this group include chronic bronchitis, asthmatic bronchitis, and emphysema. While a number of therapeutic interventions are used and have been proposed, none are completely effective, and chronic obstructive pulmonary disease remains the fourth most common cause of death in the United States.

Lung function in patients suffering from some forms of chronic obstructive pulmonary disease can be improved by reducing the effective lung volume, typically by resecting diseased portions of the lung. Resection of diseased portions of the lungs both promotes expansion of the non-diseased regions of the lung and decreases the portion of inhaled air which goes into the lungs but is unable to transfer oxygen to the blood. Lung reduction is conventionally performed in open chest or thoracoscopic procedures where the lung is resected, typically using stapling devices having integral cutting blades. Although these procedures appear to show improved patient outcomes and increased quality of life, the procedure has several major complications, namely air leaks, respiratory failure, pneumonia and death. Patients typically spend approximately 5-7 days in post-op recovery with the majority of this length of stay attributed to managing air leaks created by the mechanical resection of the lung tissue.

In an effort to reduce such risks and associated costs, minimally or non-invasive procedures have been developed. Endobronchial Volume Reduction (EVR) allows the physician to use a catheter-based system to reduce lung volumes. With the aid of fiberoptic visualization and specialty catheters, a physician can selectively isolate a segment or segments of the diseased lung. A pulmonary device that is occlusive, valved, or flow restrictive, is implanted within the lung segment to isolate a diseased region of a lung to cause the lung segment to collapse via atelectasis (collapse of the lung). By creating areas of selective atelectasis, the total lung volume is reduced and the patient's breathing mechanics is enhanced by creating more space inside the chest wall cavity for the healthy segments to function more efficiently.

Occasionally however, pulmonary devices intended to create atelectasis, which may also be referred to as pulmonary stents, may be poorly positioned, move, leak, dislodge, cause irritation, or may otherwise be dysfunctional and need to be removed. Conventional biopsy graspers may be used for this purpose. However, these graspers are sometimes less than ideal and are ineffective because the jaws of such graspers cannot open wide enough or may create a risk of potential injury to the bronchial wall. Therefore, more effective tools and methods for removing pulmonary stents are desired.

2. Description of Background Art

Implanted devices of the type removed by the methods and devices of the present invention are described in U.S. Pat. No. 6,527,761 (Attorney Docket No. 017534-001200US), U.S. patent application Ser. No. 11/280,592 (Attorney Docket No. 017534-002010US), and U.S. patent application Ser. No. 11/280,530 (Attorney Docket No. 017534-002110US), each of which are commonly assigned to the assignee of the present application and are incorporated herein by reference. Implant removal tools are described in U.S. Pat. No. 7,165,584; U.S. Publication No. 2004/089306 A1; and U.S. Publication No. 2006/162,731 A1.

### BRIEF SUMMARY OF THE INVENTION

Generally, the present invention provides devices and methods for removing implanted devices, such as pulmonary stents and related devices which may be occlusive, valved or flow-restrictive, from a body lumen, such as a bronchial passage.

In one aspect of the invention, a removal apparatus comprises an elongate tube having a central passage, an inner member slideably disposed within the passage, and a coupling member disposed near the distal end of the inner member. The removal apparatus is adapted to remove a removeably implanted devices. Removeably implanted devices include pulmonary stents, occlusive devices, valved devices, and flow-restrictive devices. Such devices often comprise a support element or frame and a membrane which may at least partially cover the support element. The coupling member of the removal apparatus is adapted to releasably engage the support element and/or the membrane.

In many embodiments, the coupling member comprises a rotatable coil and a distal tip. The distal tip is adapted to pierce the membrane of the implanted device and often does so as the inner member of the removal apparatus is advanced through the passage distally towards the implanted device. The distal tip is often within the axial profile of the coil. The coil may be integral with the distal tip. The distal tip may be a straight tip, an open loop of the coil, a hook, or the like. In the case where the distal tip is an open loop of the coil, the open loop is often configured to engage the support element of the implanted device.

In some embodiments, the coupling member comprises a hook which is separately moveable from the distal tip.

In some embodiments, the loops of the coil are adapted to engage the support element of the implanted device as it is rotated.

In some embodiments, the coil comprises a heat-shrink covering to reduce friction.

In some embodiments, the coil further comprises a torque transmission element to improve the torque transmission abilities of the coil. The torque transmission element may be a counter-wound concentric coil, an axial wire, an axial strand, a suture or the like.

In many embodiments, the inner member comprises a shaft and the coupling member comprises a hook disposed on the distal end of the shaft. The hook is often adapted to pierce the membrane of the implanted device and engage the support element of the implanted device as the inner member is advanced distally towards the implanted device. As with the previously described hooks, the hook may be J-shaped, C-shaped or G-shaped.

In many embodiments, the coupling member comprises a tube having a number of notches. The notches are often is configured to engage the support element of the implanted device without requiring rotation of the coil. Additionally, the distal end of the tube is adapted to pierce the membrane of the implanted device as the inner member is advanced through the passage distally towards the implanted device.

In a further aspect of the invention, a method is provided for removing a removeably implanted device positioned within a body lumen. The removeably implanted device may be a pulmonary stent, an occlusive device, a valved device, or a flow-restrictive device. The implanted device often comprises a frame at least partially covered by a membrane. The removal tool is positioned so that the distal end of the removal tool is adjacent to the implanted device. The distal end of the removal tool pierces the membrane of the implanted device, often by advancing the distal end of the removal tool distally towards the implanted device. The removal tool then captures an element of the frame. The distal end of the removal tool is subsequently retracted along with the implanted device.

In many embodiments, the removal tool comprises a rotatable coil and the removal tool captures the element of the frame by rotating the coil to engage its loops with the frame element. In some embodiments, an open loop of the rotatable coil is disposed near the distal end of the removal tool and the removal tool captures the frame element by engaging it with the open loop.

In many embodiments, the removal tool comprises a tube disposed near its distal end. The tube comprises a number of notches and the removal tool captures the frame element by engaging it with the notches.

In many embodiments, the removal tool comprises a hook disposed near its distal end. The removal tool captures the frame element by engaging it with the hook. In some embodiments, the hook is separately moveable from the distal end of the removal tool and hook may separately lock with the frame element to capture it.

### BRIEF DESCRIPTION OF THE DRAWINGS

FIG. 1 is a cross-sectional view of a lung having implants positioned therein.

FIG. 2A shows a pulmonary stent.

FIG. 2B shows the pulmonary stent of FIG. 2A positioned within a bronchial passage.

FIG. 3 shows a removal device embodiment of the present invention.

FIG. 4A-4D show a method of removing an implant using the device shown in FIG. 3.

FIG. 5A-5D show alternative distal structures which may be incorporated in the removal devices of the present invention.

FIG. 6 shows an alternative embodiment of the removal device of the present invention.

FIG. 7A-7C show a method of removing an implant using the device shown in FIG. 6.

FIG. 8 shows a further embodiment of the removal device of the present invention.

FIG. 9 shows a still further embodiment of the removal device of the present invention.

FIG. 10A-10C show a method of removing an implant using the device shown in FIG. 9.

### DETAILED DESCRIPTION OF THE INVENTION

Removal tools for pulmonary devices, particularly occlusive, valved or flow-restrictive devices and stents that are used for atelactasis, are disclosed. However, it may be appreciated that the removal tool may be used to remove various other types of devices that have been positioned in a variety of bodily passageways, such as blocking devices in the fallopian tubes and the like.

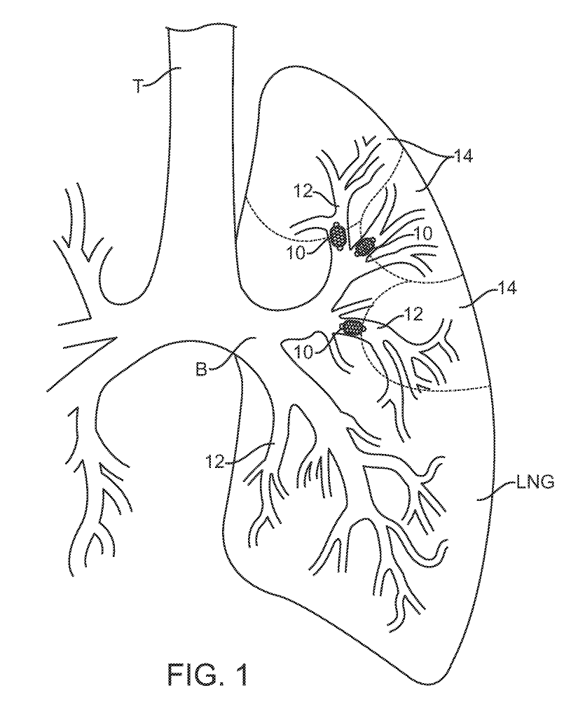

FIG. 1 shows a cross-section of the right lung LNG fed by bronchus B and trachea T. Removeably implanted devices 10 have been positioned within various bronchial passages 12. As shown in FIG. 1, implanted devices 10 are pulmonary devices which occlude the areas of the lung distal of where they are placed, reducing lung volume typically by inducing atelectasis in segments 14. Alternatively, pulmonary devices could restrict air flow in the inhalation direction and permit air flow in the exhalation direction out of the isolated segments 14 eventually leading to atelectasis of segments 14.

Methods and devices for achieving such segmental isolation and volume reduction using occlusive, valved and flow restrictive pulmonary stents are described in U.S. Pat. Nos. 6,527,761 (Attorney Docket No. 017534-001200US) and 6,997,918 (Attorney Docket No. 017534-001210US), as well as in commonly owned, pending application Ser. Nos. 11/280,592 (Attorney Docket No. 017534-002010US); 11/280,530 (Attorney Docket No. 017534-002110US); and 11/682,986 (Attorney Docket No. 017534-003510US), the full disclosures of which are incorporated herein by reference.

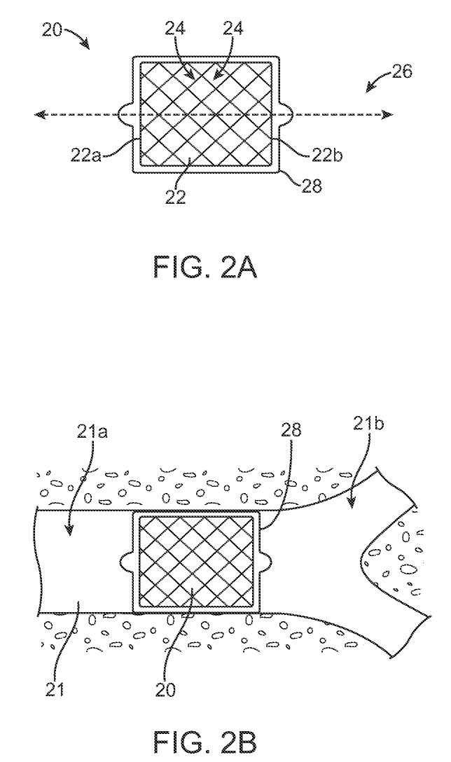

FIG. 2A is a detailed, cross-sectional view of pulmonary device or stent 20. FIG. 2B shows pulmonary stent 20 placed within a bronchial passage 21 and occluding an area of the lung distal to the stent (to the right in FIG. 2B). Pulmonary stent 20 includes support member 22, usually an expansible, tubular scaffold or frame composed of a number of struts 24. As shown, struts 24 are arranged in a braided pattern. However, struts 24 may be arranged in other patterns as well, for example each of struts 24 may be parallel to longitudinal axis 26 of support member 22. Support member 22 provides mechanical support to occlude a bronchial passage and to keep itself in position when placed therein. Support member 22 is often made of a resilient material, for example a shape memory alloy such as Nitinol™. Pulmonary stent 20 also includes an occlusive membrane 28 which completely surrounds support member 22, including its two ends 22a and 22b. Membrane 28 is often thin and resilient and may be coated on support member 22. Prior to placement at a particular location within the lung, pulmonary stent 20 is often radially compressed about axis 26 to allow for placement in a delivery system, which can be delivered through the bronchoscope near the vicinity of a the target passage. Once in position and upon activation of the delivery system, pulmonary stent 20 is allowed to radially expand, occluding the targeted lung area and maintaining stent 20 in position. Although support member 22 may be hollow, membrane 28 which surrounds support member 22 forms an occlusive seal, isolating the portion 21a of the bronchial passage proximal to the pulmonary stent 20 from the portion 21b of the bronchial passage that is distal to pulmonary stent 20.

As shown in FIGS. 2A and 2B, pulmonary stent 20 is fully occlusive, i.e., all flow in either direction is blocked. Other pulmonary devices that are used for inducing atelectasis are based on restricting fluid flow in one direction using valves or other types of flow-restrictive devices, which are described in the applications and patents referenced earlier. Valved stents will carry a one-way valve where the stent is positioned to allow air to flow out of but not into the isolated lung segment. Flow restrictive stents allow low bi-directional air flow in and out of the isolated segment to provide for a controlled atelectasis as described in application Ser. No. 11/682,986 (Attorney Docket No. 017534-003510US). The removal tools of the present invention may be used with all the different types of pulmonary devices and stents.

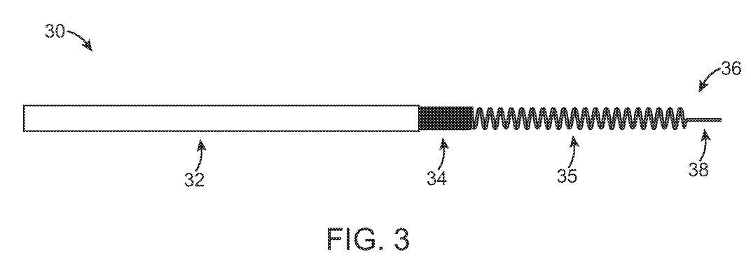

FIG. 3 shows a pulmonary stent removal device 30 according to the present invention. Removal device 30 includes a elongate tube 32, often in the form of a catheter 32 configured to pass through a bronchoscope to access a desired bronchial passage. Inner member 34 is slideably disposed within tube or catheter 32. In the embodiment shown in FIG. 3, inner member 34 comprises a coil 35, which may be rotated about to the axis of tube 32. A distal tip or straight point 38 is disposed on the distal end of inner member 34. Distal tip or straight point 38 is often integral with coil 35 and is sufficiently sharp to penetrate the membrane of a pulmonary stent.

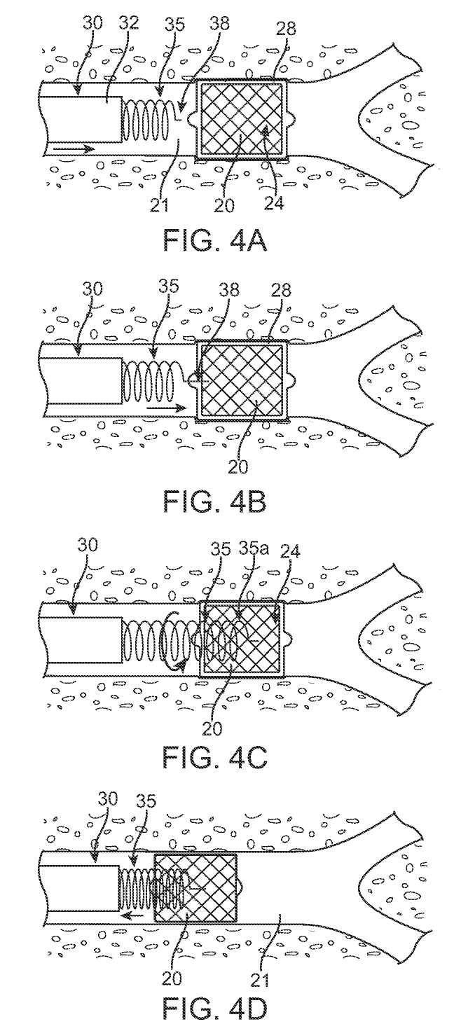

FIGS. 4A-4D show a method of removing pulmonary stent 20, shown in FIGS. 2A and 2B, from bronchial passage 21 using removal device 30, shown in FIG. 3. Removal device 30 is navigated through a bronchoscope to access the desired area in the bronchus—the location of pulmonary stent 20. As shown in FIG. 4A, removal device 30 is first steered by a bronchoscope to a position immediately adjacent to pulmonary stent 20. As shown in FIG. 4B, coil 35 is then advanced distally so that straight tip 38 pierces membrane 28. Afterwards, as shown in FIG. 4C, coil 35 is rotated, either clockwise or counterclockwise. The rotation of coil 35 causes the loops 35a of coil 35 to capture or engage struts 24 of pulmonary stent 20, engaging stent 20 onto coil 35. Optionally, a heat-shrink covering may be added to coil 35 to reduce friction during rotation and delivery of the coil. Additionally, either some or all of the coil may incorporate counter-wound concentric coils or an axial wire, strand, or suture to improve the torque transmission capabilities of the coil. Once removal device 30 is engaged with pulmonary stent 20, coil 35 may then be retracted to retrieve pulmonary stent 20, as shown in FIG. 4D.

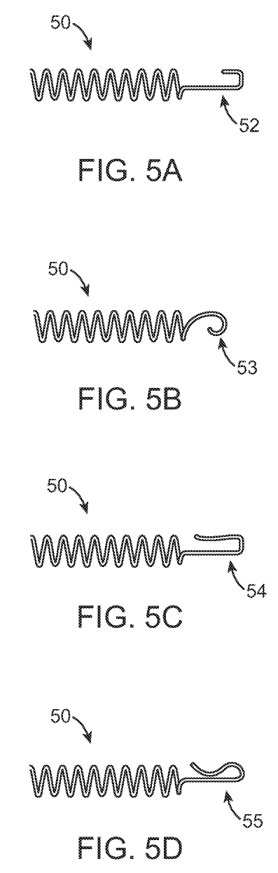

FIGS. 5A-5D show alternative distal ends 50 which may be incorporated in the removal devices of the present invention. Distal ends 50 are often within the axial profile of the coil so they can easily slide through a delivery catheter and be non-traumatic to the airway. Although the removal device is similar that those previously described with reference to FIG. 3, instead of a straight point extending from the distal end of the coil of the removal device, a hook may be present. The hook will usually be integral with the coil of the removal device but could be formed separately and attached. The hook allows the device to capture one of the struts of a pulmonary stent. With the hook, rotation of the coil is not required but could be useful to further lock the stent onto the coil. The hook may be a straight, J-shaped hook 52 as shown in FIG. 5A. Alternatively, the hook may be minimally straight and instead be substantially curved, for example, the C-shaped hook 53 of FIG. 5B. FIG. 5C shows yet another hook that may be used—a sturdy J-shaped hook 54, which is generally similar to J-shaped hook 52 but has a relatively longer end. FIG. 5D shows yet another hook that may be used. G-shaped hook 55 is generally similar to J-shaped hook 51 but has a curved end. The curved end of G-shaped hook 55 allows the hook to positively attach to the pulmonary stent during retrieval of the stent using the removal device.

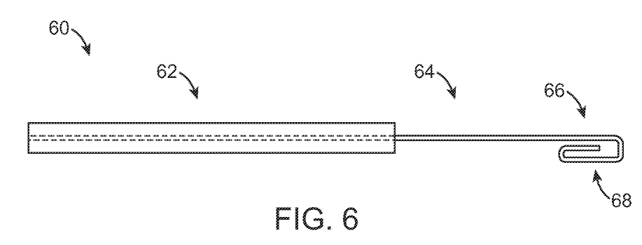

FIG. 6 shows another embodiment of a pulmonary stent removal device according to the present invention. Removal device 60 is similar to previously described removal device 30. Removal device 60 includes catheter 62, an inner member 64 slideably disposed within catheter 62, and coupling member 68 disposed on the distal end 66 of inner member 64. However, inner member 64 does not comprise a rotatable coil but instead includes a shaft 64 which is strongly resistant to axial compression. Removal device 60 relies on coupling member 68 to engage a pulmonary stent. Coupling member 68 may be a G-shaped hook as shown or be another type hook generally similar to those described above with reference to FIGS. 5A-5D.

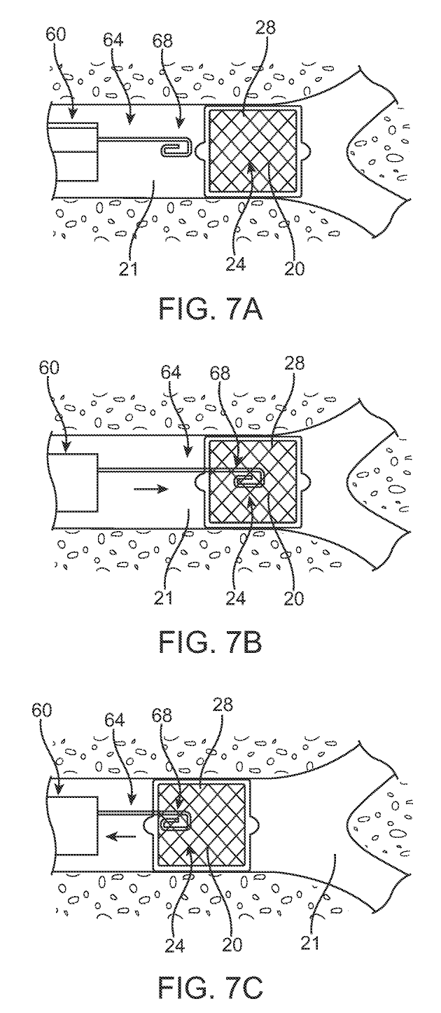

FIGS. 7A-7C show a method of removing pulmonary stent 20 within bronchial passage 21 using removal device 60 of FIG. 6. The method is generally similar to the method described above with reference to FIGS. 4A-4B. However, rotation of a coil is not required to couple the removal device with the pulmonary stent. As shown in FIG. 7A, removal device 60 is positioned immediately adjacent to pulmonary stent 20. As shown in FIG. 7B, shaft 64 is then advanced distally until it pierces membrane 28 of pulmonary stent 20. Coupling member 68 captures at least one of struts 24. This allows the proximal movement of shaft 64 to retrieve pulmonary stent 20, as shown in FIG. 7C.

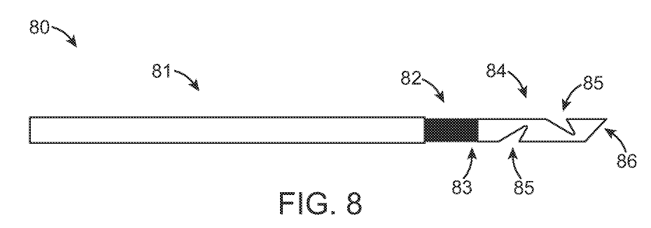

FIG. 8 shows a pulmonary stent removal device according to other embodiments of the present invention. Removal device 80 is similar to previously described removal devices 30 and 60. Removal device 80 includes a catheter 81, inner member 82 slideably disposed within catheter 81, and coupling member 84 disposed on the distal end 83 of catheter 81. Inner member 82 is often a coil. Coupling member 84 is in the form of a tube 84 having a number of sharp indents or notches (cuts) 85. As shown in FIG. 8, tube 84 has two notches 85 although tube 84 may have any number of notches 85, for example three or more. Notches 85 are configured to capture or engage a pulmonary stent without requiring rotation. After engagement, catheter 81 may be moved against the stent for more dependable attachment. Distal end 86 of coupling member 84 is sharp and allows the removal device 80 to pierce the membrane of a pulmonary stent. A method similar to the methods previously described may be used to remove a pulmonary stent using removal device 80.

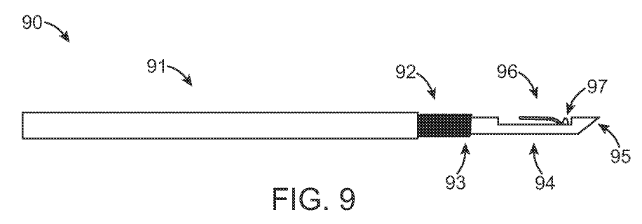

FIG. 9 shows a pulmonary stent removal device according to further embodiments of the present invention. Removal device 90 is generally similar to removal previously described devices 30, 60 and 80. Removal device 90 includes a catheter 91, inner member 92 slideably disposed within catheter 91, and coupling member 93 disposed on the distal end 94 of inner member 92. Inner member 92 may comprise a coil. Distal end 95 of coupling member 93 is sharp and allows the removal device 90 to pierce the membrane of a pulmonary stent. Coupling member 93 includes hook 96. Hook 96 may be J-shaped. Optionally, hook 96 is separately movable from coupling member 93. For example, a retainer 97 may be provided to retain hook 96 in place. Hook 96 may be made of a resilient material such as Nitinol™. Retainer 97 may be actuated to place hook 96 in an expanded shape or to retract it back to a contracted shape. This allows hook 96 to be moved proximally to capture and lock a strut of the pulmonary stent against the proximal portion of removal device 90 to prevent detachment of removal device 90 from the pulmonary stent.

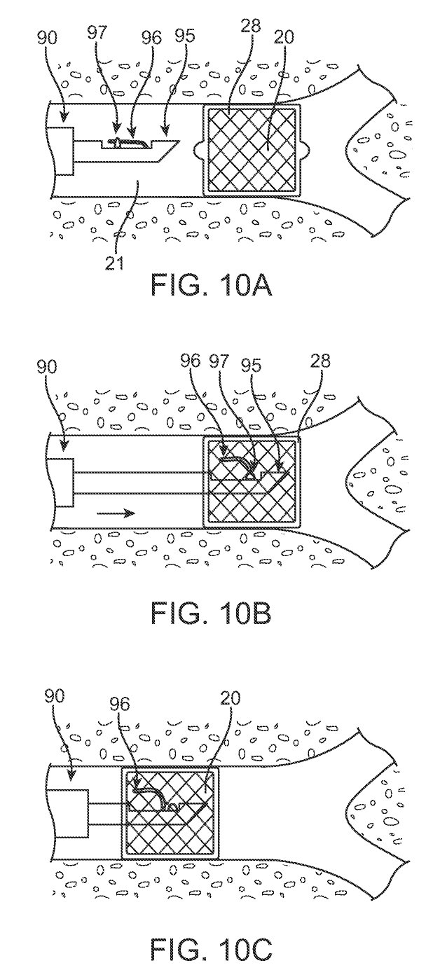

As shown in FIGS. 10A-10C, a method similar to those previously described may be used to remove pulmonary stent 20 using removal device 90 of FIG. 9 from a bronchial passage 21. Removal device 90 is placed adjacent to the proximal end of a pulmonary stent and advanced distally. Sharp distal end 95 pierces the membrane 28 of the pulmonary stent. Retainer 97 is pushed forward, allowing hook 96 to expand. Retraction of removal device 90 causes hook 96 to capture at least one of the struts of the pulmonary stent thereby allowing retrieval of the stent. Retainer 97 may be pulled back to cause hook 96 to contract and lock onto the captured or engaged struts. Although rotation of inner member 92 is not required, it could be useful to further lock the stent onto the coil. Distal end 95 is then retracted along with pulmonary stent 20.

While the above is a complete description of various embodiments of the present invention, it will be appreciated by those skilled in the art that various alternatives, modifications and equivalents may be used without departing from the spirit and scope of the present invention, which is solely limited by the appended claims.

What is claimed is:

1. An apparatus for removing a removeably implanted device positioned within a body lumen, the apparatus comprising:
an elongate tube having a central passage;
an inner member slideably disposed within the passage of the elongate tube; and
a coupling member disposed near a distal end of the inner member, said coupling member adapted to releasably engage a support element and/or a membrane of the implanted device.

2. The apparatus of claim 1, wherein the coupling member comprises a rotatable coil and a distal tip, the distal tip being adapted to pierce the membrane of the implanted device as the inner member is advanced through the passage distally towards the implanted device.

3. The apparatus of claim 2, wherein the axial profile of the distal tip is within the axial profile of the coil.

4. The apparatus of claim 2, wherein the coil is integral with the distal tip.

5. The apparatus of claim 2, wherein the distal tip comprises a straight tip.

6. The apparatus of claim 2, wherein the distal tip comprises an open loop of the coil, the open loop being configured to engage the support element of the implanted device.

7. The apparatus of claim 2, wherein the distal tip comprises a hook, the hook being configured to engage the support element of the implanted device.

8. The apparatus of claim 2, wherein the coupling member comprises a hook, the hook being configured to engage the support element of the implanted device and separately moveable from the distal tip.

9. The apparatus of claim 2, wherein the loops of the coil are adapted to engage the support element of the implanted device as the coil is rotated.

10. The apparatus of claim 2, wherein the coil comprises a heat-shrink covering to reduce friction.

11. The apparatus of claim 2, wherein the coil comprises a torque transmission element.

12. The apparatus of claim 11, wherein the torque transmission element comprises a counter-wound concentric coil.

13. The apparatus of claim 11, wherein the torque transmission element comprises an axial wire.

14. The apparatus of claim 11, wherein the torque transmission element comprises an axial strand.

15. The apparatus of claim 11, wherein the torque transmission element comprises a suture.

16. The apparatus of claim 1, wherein the inner member comprises a shaft and the coupling member comprises a hook disposed on the distal end of the shaft, the hook being adapted to pierce the membrane of the implanted device and engage the support element of the implanted device as the inner member is advanced distally towards the implanted device.

17. The apparatus of claims 7, 8 or 16, wherein the hook comprises a J-shaped hook.

18. The apparatus of claims 7, 8 or 16, wherein the hook comprises a C-shaped hook.

19. The apparatus of claims 7, 8 or 16, wherein the hook comprises a G-shaped hook.

20. The apparatus of claim 1, wherein the coupling member comprises a tube having at least one notch, wherein the at least one notch is configured to engage the support element of the implanted device without requiring rotation of the coil and a distal end of the tube is adapted to pierce the membrane of the implanted device as the inner member is advanced through the passage distally towards the implanted device.

21. A method for removing a removeably implanted device positioned within a body lumen, the implanted device comprising a frame at least partially covered by a membrane, the method comprising:
positioning a removal tool so that a distal end of the removal tool is adjacent to the implanted device;
piercing the membrane of the implanted device with the distal end of the removal tool;
capturing an element of the frame with the removal tool; and
retracting the distal end of the removal tool along with the implanted device.

22. The method of claim 21, wherein piercing the membrane of the implanted device with the distal end of the removal tool comprises advancing the distal end of the removal tool distally towards the implanted device.

23. The method of claim 21, wherein the removal tool comprises a rotatable coil; and
capturing an element of the frame with the removal tool comprises rotating the coil to engage the loops of the coil with the frame element.

24. The method of claim 23, wherein an open loop of the rotatable coil is disposed near the distal end of the removal tool; and
capturing an element of the frame with the removal tool comprises engaging the open loop with the frame element.

25. The method of claim 21, wherein the removal tool comprises a tube having at least one notch disposed near the distal end of the removal tool; and
capturing an element of the frame with the removal tool comprises engaging the at least one notch with the frame element.

26. The method of claim 21, wherein the removal tool comprises a hook disposed near the distal end of the removal tool; and
capturing an element of the frame with the removal tool comprises engaging the hook with the frame element.

27. The method of claim 26, wherein the hook is separately moveable from the distal end of the removal tool; and
capturing an element of the frame with the removal tool further comprises locking the hook with the frame element.

28. The method of claim 21, wherein the implanted device is a pulmonary stent.

29. The method of claim 21, wherein the implanted device is an occlusive device.

30. The method of claim 21, wherein the implanted device is a valved device.

31. The method of claim 21, wherein the implanted device is a flow-restrictive device.
